(19) United States
(12) Patent Application Publication
(10) Pub. No.: US 2006/0078599 A1
(43) Pub. Date: Apr. 13, 2006

(54) Pharmaceutical composition applicable to body tissue

(76) Inventors: Mathew Ebmeier, Fort Collins, CO (US); Robert Lathrop, Fort Collins, CO (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/962,989

(22) Filed: Oct. 12, 2004

(51) Int. Cl. A61K 36/14; A61K 31/717; A61K 31/137; A61K 31/045; A61K 31/125

(52) U.S. Cl. 424/443; 514/57; 514/649; 424/770; 514/729; 514/692; 514/730; 514/731; 424/725.1

(57) ABSTRACT

The present invention provides a non-water soluble, film-forming composition which adheres to body tissue and forms a pharmaceutical carrier to provide localized delivery of an antifungal agent to a treatment site. The composition will typically include: (a) an alkyl cellulose; (b) a hydroxyalkyl cellulose; (c) a pharmaceutically acceptable polar protic solvent; (d) an antifungal agent selected from the group of naftifine, ciclopirox, terbinafine, pharmaceutically acceptable salts thereof, and combinations thereof; (e) an glycol ether; (f) an antipruritic agent selected from the group of camphor, menthol, butamben picrate, metacresol, benzyl alcohol, camphorated metacresol, juniper tar, phenol, phenolate sodium, resorcinol, camphorated metacresol, carbolic acid, and combinations,; and (g) a solubility enhancing agent, a surfactant, a wetting agent, or a combination thereof. The present invention also provides for the use of the composition composition of the present invention, in treating a fungal infection (e.g., nail fungus) in a mammal afflicted with such an infection.

### BACKGROUND OF THE INVENTION

The localized treatment of body tissues, diseases, and wounds requires that the particular pharmaceutical component be maintained at the site of treatment for an effective period of time. On topical surfaces (e.g., the skin and nails), sweat, clothing and movements are some of the problems that have limited the effectiveness and residence time of pharmaceutical carriers. As such, what is needed is a composition that adheres to body tissue, and provides localized delivery of a pharmaceutical to the body tissue. More specifically, what is needed is a composition that adheres to skin and nail surfaces, and provides localized delivery of an antifungal agent to a nail and surround skin surfaces that are afflicted with a fungal infection.

### SUMMARY OF THE INVENTION

The present invention provides a non-water soluble, film-forming composition which adheres to body tissue and forms a pharmaceutical carrier to provide localized delivery of an antifungal agent to a treatment site. The composition includes: (a) an alkyl cellulose present in about 0.5% (w/w) to about 3.0% (w/w) of the composition; (b) a hydroxyalkyl cellulose present in about 2.0% (w/w) to about 10.0% (w/w) of the composition; (c) a pharmaceutically acceptable polar protic solvent present in about 30.0% (w/w) to about 95.0% (w/w) of the composition; (d) an antifungal agent selected from the group of naftifine, ciclopirox, terbinafine, pharmaceutically acceptable salts thereof, and combinations thereof, present in about 2.5% (w/w) to about 15% (w/w) of the composition; (e) an glycol ether present in about 4.0% (w/w) to about 12.0% (w/w) of the composition; (f) an antipruritic agent selected from the group of camphor, menthol, butamben picrate, metacresol, benzyl alcohol, camphorated metacresol, juniper tar, phenol, phenolate sodium, resorcinol, camphorated metacresol, carbolic acid, and combinations, present in about 1.0% (w/w) to about 5.0% (w/w) of the composition; and (g) a solubility enhancing agent, a surfactant, a wetting agent, or a combination thereof, present in about 1.0% (w/w) to about 3.0% (w/w) of the composition.

The present invention also provides a non-water soluble, film-forming composition which adheres to body tissue and forms a pharmaceutical carrier to provide localized delivery of naftifine, or a pharmaceutically acceptable salt thereof, to a treatment site. The composition includes: (a) ethyl cellulose; (b) hydroxypropyl cellulose; (c) purified water; (d) ethanol; (e) naftifine, or a pharmaceutically acceptable salt thereof; (f) diethylene glycol monoethyl ether (DGME); (g) benzyl alcohol; and (h) sodium laureth ether sulfate (SLES).

The present invention also provides a non-water soluble, film-forming composition which adheres to body tissue and forms a pharmaceutical carrier to provide localized delivery of naftifine, or a pharmaceutically acceptable salt thereof, to a treatment site. The composition includes: (a) ethyl cellulose present in about 1.0% (w/w) of the composition; (b) hydroxypropyl cellulose present in about 5.0% (w/w) of the composition; (c) purified water present in about 6.0% (w/w) of the composition; (d) ethanol 190 present in about 70.0% (w/w) of the composition; (e) naftifine, or a pharmaceutically acceptable salt thereof, present in about 5.0% (w/w) of the composition; (f) diethylene glycol monoethyl ether (DGME) present in about 8.0% (w/w) of the composition; (g) benzyl alcohol present in about 3.0% (w/w) of the composition; and (h) sodium laureth ether sulfate (SLES) present in about 2.0% (w/w) of the composition.

The present invention also provides a non-water soluble, film-forming composition which adheres to body tissue and forms a pharmaceutical carrier to provide localized delivery of naftifine, or a pharmaceutically acceptable salt thereof, to a treatment site. The composition includes: (a) ethyl cellulose present in about 1.0% (w/w) of the composition; (b) hydroxypropyl cellulose present in about 5.0% (w/w) of the composition; (c) purified water present in about 6.0% (w/w) of the composition; (d) ethanol 190 present in about 65.0% (w/w) of the composition; (e) naftifine, or a pharmaceutically acceptable salt thereof, present in about 10.0% (w/w) of the composition; (f) diethylene glycol monoethyl ether (DGME) present in about 8.0% (w/w) of the composition; (g) benzyl alcohol present in about 3.0% (w/w) of the composition; and (h) sodium laureth ether sulfate (SLES) present in about 2.0% (w/w) of the composition.

The present invention also provides a non-water soluble, film-forming composition which adheres to body tissue and forms a pharmaceutical carrier to provide localized delivery of naftifine, or a pharmaceutically acceptable salt thereof, to a treatment site. The composition consists essentially of: (a) ethyl cellulose; (b) hydroxypropyl cellulose; (c) purified water; (d) ethanol; (e) naftifine, or a pharmaceutically acceptable salt thereof; (f) diethylene glycol monoethyl ether (DGME); (g) benzyl alcohol; and (h) sodium laureth ether sulfate (SLES).

The present invention also provides a non-water soluble, film-forming composition which adheres to body tissue and forms a pharmaceutical carrier to provide localized delivery of naftifine, or a pharmaceutically acceptable salt thereof, to a treatment site. The said composition consists essentially of: (a) ethyl cellulose present in about 1.0% (w/w) of the composition; (b) hydroxypropyl cellulose present in about 5.0% (w/w) of the composition; (c) purified water present in about 6.0% (w/w) of the composition; (d) ethanol 190 present in about 70.0% (w/w) of the composition; (e) naftifine, or a pharmaceutically acceptable salt thereof, present in about 5.0% (w/w) of the composition; (f) diethylene glycol monoethyl ether (DGME) present in about 8.0% (w/w) of the composition; (g) benzyl alcohol present in about 3.0% (w/w) of the composition; and (h) sodium laureth ether sulfate (SLES) present in about 2.0% (w/w) of the composition.

The present invention also provides a non-water soluble, film-forming composition which adheres to body tissue and forms a pharmaceutical carrier to provide localized delivery of naftifine, or a pharmaceutically acceptable salt thereof, to a treatment site. The composition consists essentially of: (a) ethyl cellulose present in about 1.0% (w/w) of the composition; (b) hydroxypropyl cellulose present in about 5.0% (w/w) of the composition; (c) purified water present in about 6.0% (w/w) of the composition; (d) ethanol 190 present in about 65.0% (w/w) of the composition; (e) naftifine, or a pharmaceutically acceptable salt thereof, present in about 10.0% (w/w) of the composition; (f) diethylene glycol monoethyl ether (DGME) present in about 8.0% (w/w) of the composition; (g) benzyl alcohol present in about 3.0% (w/w) of the composition; and (h) sodium laureth ether sulfate (SLES) present in about 2.0% (w/w) of the composition.

The present invention also provides a method for inhibiting a fungus. The method includes contacting a fungus with an effective amount of the composition of the present invention.

The present invention also provides a method for treating a fungal infection in a mammal in need of such treatment. The method includes contacting a fungal infection with an effective amount of the composition of the present invention.

### DETAILED DESCRIPTION OF THE INVENTION

The compositions of the invention are useful for treating or preventing fungal infections, e.g., nail fungus. Infections cause by dermatophytes (e.g., molds and fungus) for which the compositions of the present invention may have activity include, but are not limited to: Ascomycetes (e.g., Neurospora, Saccharomyces, Morchella), Basidiomycetes (e.g., Amanita, Agaricus), Zygomycetes (e.g., Mucor, Rhizopus), Oomycetes (e.g., Allomyces), and Deuteromycetes (e.g., Penicillium, Aspergillus), Microsporum canis, Microsporum gypseum, Microsporum audouinii, Trichophyton tonsurans, Trichophyton mentagrophytes, Epidermophyton floccosum, and Trichophyton rubum, Candida albicans, Candida guilliermondii, Epidermophyton (e.g., floccosum), Microsporum (e.g., canis), Trichophyton (e.g., rubrum), Acremonium spp., Aspergillus spp., Candida spp., Fusarium (e.g., oxysporum), Scopulariopsis (e.g., brevicaulis), Onychocola (e.g., Canadensis), Scytalidium (e.g., dimidatum), and combinations thereof.

Definitions:

The following definitions are used, unless otherwise described: alkyl, denotes both straight and branched groups; but reference to an individual radical such as “propyl” embraces only the straight chain radical, a branched chain isomer such as “isopropyl” being specifically referred to. When alkyl can be partially unsaturated, the alkyl chain may comprise one or more (e.g. 1, 2, 3, or 4) double or triple bonds in the chain.

As used herein, “non-water soluble” refers to a solubility in water, at ambient conditions, of less than about 1% (w/w). Specifically, the term refers to a solubility in water, at ambient conditions, of less than about 0.5% (w/w) or less than about 0.1% (w/w).

As used herein, “film-forming” refers to the ability of a substance to form a film at ambient conditions.

As used herein, “composition” refers to the solid or semisolid phase of a colloidal solution usually containing medicinal substances and intended for external application. Stedman's Medical Dictionary, Williams & Wilkins, Baltimore, Md., 25th Edition, (1990). The term composition refers to a compositionatinous; jelly-like colloid. Concise Chemical and Technical Dictionary, Chemical Publishing Co., Inc., New York, N.Y., Fourth Enlarged Edition, (1986).

As used herein, “body tissue” refers to any group of cells that perform specific functions. A tissue need not form a layer. Thus, bone marrow is a tissue; connective tissue consists of cells that make up fibers in the framework supporting other body tissues; and lymphoid tissue is the part of the body's immune system that helps protect it from bacteria and other foreign entities. Specifically, the tissue can include skin (dennis), fingernails, toenails, or any combination thereof.

As used herein, “antifungal agent” or “fungicide” refers to a chemical that will kill, destroy, inhibit, or inactivate a fungus to prevent growth. The chemical can be synthetic or biosynthetic and can include both organic and inorganic compounds. The fungicide can be a solid (e.g., powder), liquid, or a combination thereof. See, e.g., Concise Chemical and Technical Dictionary, Fourth Enlarged edition, Bennett, Chemical Publishing Company, NY, N.Y. (1986); and McGraw-Hill Concise Encyclopedia of Science & Technology, Fourth Edition, Parker, McGraw-Hill, NY, N.Y., (1998). Specifically, “fungicide” or “antifungal agent” can include a chemical that will kill, destroy, inhibit, or inactivate a eucaryotic microorganism to prevent growth. Exemplary eucaryotic microorganisms include algae, fungi, slime mold, protozoa, and eucaryotes in the microbial world. Specifically, the fungi can include, e.g., Ascomycetes (e.g., Neurospora, Saccharomyces, Morchella), Basidiomycetes (e.g., Amanita, Agaricus), Zygomycetes (e.g., Mucor, Rhizopus), Oomycetes (e.g., Allomyces), and Deuteromycetes (e.g., Penicillium, Aspergillus), Microsporum canis, Microsporum gypseum, Microsporum audouinii, Trichophyton tonsurans, Trichophyton mentagrophytes, Epidermophyton floccosum, and Trichophyton rubum, Candida albicans, Candida guilliermondii, or any combination thereof.

As used herein, “alkyl cellulose” refers to a substituted poly-alkyl ether of cellulose. The alkyl side chains may be straight chained (e.g., methyl, ethyl, or n-propyl) or branched (e.g., tert-butyl).

As used herein, “hydroxyalkyl cellulose” refers to a substituted poly-hydroxyalkyl ether of cellulose. The hydroxyalkyl side chains may be straight chained (e.g., hydroxymethyl, hydroxyethyl, or hydroxypropyl) or branched (e.g., hydroxyl-tert-butyl).

As used herein, “polar protic solvent” refers to a liquid at ambient conditions that has an appreciable dipole and includes at least one hydrogen atom available for hydrogen bonding. Suitable polar protic solvents include, e.g., water, (C₁-C₃₀)alkyl substituted with one or more hydroxyl groups (e.g., methanol, ethanol, propanol, glycerin, etc.), acetic acid, ammonia, etc.

As used herein, “naftifine” refers to (E)-N-Cinnamyl-N-methyl-1-naphthalenemethylamine; and “naftifine hydrochloride” refers to (E)-N-Cinnamyl-N-methyl-1-naphthalenemethylamine hydrochloride.

As used herein, “ciclopirox” refers to a synthetic broad-spectrum antifungal agent that inhibits the growth of dermatophytes, a type of fungus that grows on the skin, hair, and nails. Ciclopirox was approved for use in the U.S. by the FDA in December, 1999. Ciclopirox is chemically designated as 6-cyclohexyl-1-hydroxy-4-methyl-2(1H) pyridinone.

As used herein, “terbinafine” refers to an antifungal agent that acts by interfering with the ability of fungi to make chemicals called sterols that are an important part of the membrane that surrounds fungal cells and holds them together. This weakens the cell membrane. Topical terbinafine was approved by the FDA in 1993. Terbinafine is chemically designated as (E)-N-(6,6-dimethyl-2-hepten-4-ynyl)-N-methyl-1-naphthalenemethanamine. Terbinafine hydrochloride is chemically designated as (E)-N-(6,6-dimethyl-2-hepten-4-ynyl)-N-methyl-1-naphthalenemethanamine hydrochloride.

As used herein, “glycol ether” refers to an ether formed from at least one glycol and at least one lower alkyl alcohol. Preferably the glycol is selected from an alkylene glycol such as ethylene glycol, propylene glycol, or butylene glycol. The ether portion of the glycol ether is a radical of a lower alkyl alcohol such as a C₁ to C₆ alcohol. Preferably, the ether portion alcohol is selected from methyl alcohol, ethyl alcohol, propyl alcohol, isopropyl alcohol, butyl alcohol, or isobutyl alcohol. The glycol ethers have a generalized formula of CxHyOz where x is from 4 to 10, y is from about 10 to 22, and z is from 2 to 5. According to the present invention, the glycol ethers are soluble or miscible with water and range in molecular formula from C₄ to about C₁₀.

Examples of glycol ethers under the classification of ethylene glycol ethers include ethylene glycol monopropyl ether (propoxyethanol), ethylene glycol monobutyl ether (butoxyethanol), diethylene glycol monomethyl ether (methoxydiglycol), diethylene glycol monoethyl ether (ethoxydiglycol), diethylene glycol monobutyl ether (butoxydiglycol), diethylene glycol monoisopropyl ether (isopropyldiglycol), and diethylene glycol monoisobutyl ether (isobutyl diglycol).

Glycol ethers under the classification of propylene glycol ethers include propylene glycol monomethyl ether, dipropylene glycol monomethyl ether (PPG-2 methyl ether), tripropylene glycol monomethyl ether (PPG-3 methyl ether), propylene glycol n-propyl ether, dipropylene glycol n-propyl ether (PPG-2 propyl ether), propylene glycol monobutyl ether, dipropylene glycol monobutyl ether (PPG-2 butyl ether), propylene glycol monoisobutyl ether, and dipropylene glycol dimethyl ether. In one embodiment of the invention the solvation enhancer is ethoxydiglycol. In another embodiment, the solvation enhancer is butoxydiglycol.

As used herein, “antipruritic agent” refers to a topically (i.e., externally) applied agent that prevents or relieves itching; and an “anesthetic” is a topically (i.e., externally) applied agent that can reversibly depress neuronal function, producing loss of ability to perceive pain and/or other sensations (see, Stedman's Medical Dictionary, 25th Ed., Ill., 1990, p.65, p.77, and p.99). The antipruritic agent can be any one or more of camphor, menthol, butamben picrate, metacresol, benzyl alcohol, camphorated metacresol, juniper tar, phenol, phenolate sodium, resorcinol, camphorated metacresol, and carbolic acid.

As used herein, “straight-chained (C₁-C₃₀)alkyl” refers to a linear monoradical saturated hydrocarbon chain preferably having from 1 to 30 carbon atoms, more preferably 1 to 20 carbon atoms, and even more preferably 1 to 15 carbon atoms. This term is exemplified by groups such as methyl, ethyl, n-propyl, n-butyl, n-hexyl, n-decyl, tetradecyl, stearyl, octyl, decyl, lauryl, myristyl, palmityl, and the like. The alkyl can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NRxRx or COORx, wherein each Rx is independently H or alkyl. The alkyl can optionally be interrupted with one or more non-peroxide oxy (—O—), thio (—S—), sulfonyl (SO) or sulfoxide (SO₂). The alkyl can optionally be at least partially unsaturated, thereby providing an alkenyl or alkynyl.

As used herein, “branched (C₁-C₃₀)alkyl” refers to a non-linear monoradical saturated hydrocarbon chain preferably having from 1 to 30 carbon atoms, more preferably 1 to about 20 carbon atoms, and even more preferably 1 to 15 carbon atoms. This term is exemplified by groups such as iso-propyl, iso-butyl, sec-butyl, and the like. The alkyl can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NRxRx or COORx, wherein each Rx is independently H or alkyl. The alkyl can optionally be interrupted with one or more non-peroxide oxy (—O—), thio (—S—), sulfonyl (SO) or sulfoxide (SO₂). The alkyl can optionally be at least partially unsaturated, thereby providing an alkenyl or alkynyl.

As used herein, “(C₃-C₂₀) cycloalkyl” refers to cyclic alkyl groups of from about 3 to about 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like. The cycloalkyl can optionally be substituted with one or more alkyl, alkoxy, halo, haloalkyl, hydroxy, hydroxyalkyl, aryl, heteroaryl, heterocycle, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano. The cycloalkyl can optionally be at least partially unsaturated, thereby providing a cycloalkenyl.

As used herein, “hydroxyl” refers to OH.

As used herein, “solubility enhancing agent,” “surfactant,” or wetting agent” refers to a substance capable of reducing the surface tension of a liquid in which it is dissolved. The “solubility enhancing agent,” “surfactant,” or wetting agent” also refers to a substance that increases the solubility of another substance in a specified liquid. The “solubility enhancing agent,” “surfactant,” or wetting agent” can be an ionic surfactant, a cationic surfactant, or a non-ionic surfactant.

As used herein, refers to a substance that increases the solubility of another substance in a specified solvent. Suitable solubility enhancing agents include, e.g., sodium laureth ether sulfate (SLES) and diemthyl sulfoxide (DMSO).

As used herein, “bioadhesive” refers to a substance capable of being adhesive, or imparting adhesive properties, to body tissue.

As used herein, “permeation enhancer” refers to a compound that affects the rate or amount of diffusion of a target compound into or through bodily tissue (e.g., a nail plate, skin, or mucous membrane).

As used herein, “material” or “essential significance” refers to a difference in biological activity of at least about 10% in vivo, or at least about 10% longer of a residence time of the composition in vivo.

As used herein, “purified water” refers to water (H₂O) that is at least about 99.95% (w/w) pure.

As used herein, “ethanol 190” refers to CH₃CH₂OH that includes no more than about 5% (v/v) water.

As used herein, “nail” refers to the body tissue of a human typically present on one side of the terminal portions of the fingers and toes. A toenail is produced by living skin cells in the toe. A toenail and fingernail include several parts including the nail plate (the visible part of the nail), the nail bed (the skin beneath the nail plate), the cuticle (the tissue that overlaps the plate and rims the base of the nail), the nail folds (the skin folds that frame and support the nail on three sides), the lunula (the whitish half-moon at the base of the nail) and the matrix (the hidden part of the nail unit under the cuticle). Toenails and fingernails grow from the matrix. The nails are composed larcompositiony of keratin, a hardened protein (that is also in skin and hair). As new cells grow in the matrix, the older cells are pushed out, compacted and take on the familiar flattened, hardened form of the nail. The average growth rate for nails is 0.1 mm each day (or 1 centimeter in 100 days). The exact rate of nail growth depends on numerous factors including the age and sex of the individual and the time of year. Nails generally grow faster in young people, in males, and in the summer. Fingernails grow faster than toenails. The fingernails on the right hand of a righthanded person grow faster than those on their left hand, and vice versa.

As used herein, “mammal” refers to any of the higher vertebrate animals comprising the class Mammalia, and includes, e.g., humans.

As used herein, “ambient conditions” refer to a temperature of about 65° F. to about 80° F. (e.g., 70° F.), a relative humidity of 20% to about 80% (e.g., 50%), and a pressure of about 0.85 atm. to about 1.15 atm (e.g., 1 atm.).

As used herein, “nail fungus” refers to a condition in which one of many species of fungi affect nails of a human. By far the most common species of fungi that affect nails is Trichophyton rubrum. This type of fungus that has a tendency to infect the skin (dermatophyte) and shows itself in the following specific and even peculiar ways, which are as striking as they are difficult to explain: (1) Fungal toenails are almost always accompanied by fine scaling which extends a little ways up the sides of the foot; (2) some reason, fungus rarely affects the hands unless the feet are involved first; and (3) even stranger is the striking tendency of fungus to affect only one hand at a time.

As used herein, “treating” or “treat” includes (i) preventing a pathologic condition (e.g., nail fungus) from occurring (e.g. prophylaxis); (ii) inhibiting the pathologic condition (e.g., nail fungus) or arresting its development; (iii) relieving the pathologic condition (e.g., nail fungus), and (iv) alleviating the symptoms (e.g., unsightly nails) associated with the pathologic condition (e.g., nail fungus).

In the present invention, a novel non-water-soluble composition which serves as a pharmaceutical carrier, and which adheres to topical surfaces and body tissues, is provided. One or more pharmaceutical compounds may be incorporated in the composition. The present invention finds particular use in the localized treatment of topical surfaces and body tissues such as the skin and nails. Upon application and adherence to the topical surface, the volatile or nonaqueous solvent evaporates, diffuses, or penetrates the surrounding tissues, and a film is formed. The film offers protection to the treatment site, while also providing effective drug delivery to the treatment site, surrounding body tissues, and bodily fluids. Over time, the film slowly erodes away.

The desired properties of the present invention are achieved in the combination of at least one water-insoluble, pharmacologically approved or edible alkyl cellulose or hydroxyalkyl cellulose and a volatile or nonaqueous, pharmacologically approved solvent. One or more polymers known for their bioadhesive properties may also be added to the preparation. The combination results in a non-water soluble composition which is capable of adhesion to topical surfaces or tissue. Thickening, coloring, or plasticizing agents may also be used. Upon application, the solvent evaporates, diffuses, or penetrates the surrounding tissues, and a film is formed.

The residence time of the film formed upon dissipation of the solvent depends on several factors, including the amount of composition applied, as well as the components used to make the composition and their relative percentages. Use of polymers with different molecular weights or of different chemical reactivity, for example, may affect the dissolution kinetics of the film. Residence times which have been achieved with this invention include 15 minutes to several hours, depending on the particular formulation. A preferred residence time for effective drug delivery depends on the characteristics of the particular drug, but is at least 20-30 minutes. The kinetics of drug release depend on the characteristics of the carrier composition and relative percentages of its components, the total amount of pharmaceutical incorporated into the composition, the particular application site, and the physical and chemical characteristics of the particular drug or combination of drugs.

As mentioned above, the composition of the present invention includes at least one water-insoluble, pharmacologically approved, alkyl cellulose, hydroxyalkyl cellulose, or combination thereof. Alkyl cellulose or hydroxyalkyl cellulose polymers for use in this invention include methyl cellulose, ethyl cellulose, propyl cellulose, butyl cellulose, cellulose acetate, hydroxymethyl cellulose, hydroxyethyl cellulose, hydroxypropyl cellulose, hydroxybutyl cellulose, and ethylhydroxyethyl cellulose, alone or in combination. For ethyl cellulose polymers, the preferred characteristics include an ethoxyl content between 42 and 52%, and more preferably between 44 and 50%, and for a 5% by weight of polymer in a 80/20 toluene/ethanol solution, a viscosity of between 2 and 500 cps, and more preferably between 4 and 400 cps. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as acid oleic and myristyl may be used in combination with the cellulose derivative.

The volatile, nonaqueous pharmacologically approved solvent for use in this invention should have good penetration characteristics. Some examples include low alkyl alcohols such as methanol, ethanol, and isopropyl alcohol, ethoxydiglycol, and 1 methyl-2 pyrrolidone, alone or in combination. The preferred solvent for use in this invention is a mixture of 10 to 50 parts 95% ethanol and 0 to 5 parts water, and more preferably 10 to 15 parts 95% ethanol and 1 to 3 parts water.

The antifungal agent of the present invention may include a single antifungal agent or a combination of antifungal agents. Antifungal agents which may be used, either alone or in combination, include, e.g., naftifine, ciclopirox, and terbinafine.

In addition, one or more polymers known for their bioadhesive properties may be incorporated into the composition. The polymers should be pharmacologically approved or accepted as edible components. Use of the bioadhesive polymer strengthens the adhesive nature of the film, when adhesion has to be particularly effective for reasons due to a particular drug or drug content, the specific site of application, or specific topical surface or tissue. Some polymers having bioadhesive properties for use in this invention include polyacrylic acid, cross linked or not, polyvinylpyrrolidone, and sodium carboxymethyl cellulose, alone or in combination.

Permeation enhancers may also be used to improve absorption of the drug at the treatment site. Permeation enhancers for use in this invention include sodium lauryl sulfate, sodium glycopholate, azone, EDTA, sodium cholate, sodium 5-methoxysalicylate, and others known in the art.

The relative percentages of the component materials of the present invention may vary, depending on the type of drug or combination of drugs, the particular target treatment site, the solvent, and the particular polymers used. Preferably, the solvent or combination of solvents comprises between 50 and 80% by weight of the composition. More preferably, the solvent comprises between 60 and 70% by weight. The film forming polymer or combination of polymers preferably comprises between 4 and 20% by weight of the composition, and more preferably between 6 and 12% by weight. The active pharmaceutical or combination of pharmaceuticals comprises between 0.1 and 25% by weight, more preferably between 0.2 and 20% by weight. Optionally, a bioadhesive polymer may be used and should comprise between 0 and 10% by weight, more preferably between 1 and 8% by weight. The optional flavoring, coloring, or thickening agents and/or permeation enhancer should comprise between 0 and 3% by weight, more preferably between 0.5 and 2.5% by weight.

The characteristics of the film which is formed upon application of the composition, such as thickness, tensile strength, and erosion kinetics, are not described, given that they may vary greatly depending on the properties of the tissue to which the composition is applied, the amount of composition applied, the amount of bodily fluid at the treatment site or surrounding areas, the contact surface, and other physiological factors. Because many of these physiological factors are impossible to reproduce, the mechanical properties of a film obtained ex vivo or in vitro are very different from the ones obtained in situ and have not been characterized. However, the properties of the film obtained in vivo may be adjusted via the formulation of the composition, as well as by the addition of plasticizers, the use of cross linking agents, or the amount of solvent residual.

To make the composition of the present invention, the various components are dissolved in the chosen solvent. Because of the possibility that one or more of the components might not be in solution, a suspension may also be formed. The compositionling step may take place at any moment and may be induced by the addition of a special component, a change in pH, a change in temperature, or over time. The solutions and compositions may be prepared by various methods known in the art. The composition may be applied to the treatment site by spraying, dipping, or direct application by finger or swab.

Methods for the treatment of topical surfaces using the pharmaceutical carrier of the present invention are also provided. In one embodiment, a method for the protection and localized delivery of pharmaceutical to topical surfaces comprises the steps of preparing a non-water soluble, film-forming pharmaceutical carrier having at least one water-insoluble alkyl cellulose or hydroxy alkyl cellulose, a volatile, nonaqueous solvent, and at least one active pharmaceutical component; and applying the pharmaceutical carrier to the topical surface by spraying, dipping, or direct application by finger or swab. In another embodiment, the method further comprises the use of at least one polymer having bioadhesive properties in the preparation of the pharmaceutical carrier. In a preferred embodiment, the method further comprises the use of ethyl cellulose, a 95% ethanol and water mixture; polyacrylic acid; and a local anesthetic.

### EXAMPLES

### Example 1

Naftifine In Vitro Nail Permeation

The following example provides a comparison of the in vitro nail permeation of two naftifine compositions. The 5% and 10% naftifine solutions were prepared using the specific weights of reagents illustrated in Tables 1 and 2.

TABLE 1
5% Naftifine HCl solution

| Component: | % w/w |
|---|---|
| Naftifine HCl | 5.0 |
| DGME | 8.0 |
| Benzyl Alcohol | 3.0 |
| SLES | 2.0 |
| HPC EF grade | 5.0 |
| Ethyl cellulose | 1.0 |
| Purified Water | 6.0 |
| Ethanol 190 | 70.0 |

TABLE 2
10% Naftifine HCl solution

| Component: | % w/w |
|---|---|
| Naftifine HCl | 10.0 |
| DGME | 8.0 |
| Benzyl Alcohol | 3.0 |
| SLES | 2.0 |
| HPC | 5.0 |
| Ethyl cellulose | 1.0 |
| Purified Water | 6.0 |
| Ethanol 190 | 65.0 |

The abbreviations used in Tables 1 and 2 are defined as follows: DGME is Diethylene Glycol Monoethyl Ether; SLES is Sodium Laureth Ether Sulfate; HPC is Hydroxypropyl Cellulose.

Experimental design: Five human nail plates with approximately the same size and thickness were chosen for the finite dose experiments. Each of the vertical dissolution cells was developed specifically for each nail. All finite dose applications were evaluated on the same five nails in a controlled environment of 37° C. The receptor solution was (50:25:25) acetonitrile:purified water:buffer solution (tetramethylammonium hydroxide pentahydrate (TMAHP, CAS# 10424-65-4)). Seven doses of 50 μL each were applied to simulate one week of application for each formulation. Each dose was allowed to dry prior to application of the next dose. The total dissolution time for 7 applications was approximately 7 hours. A measured aliquot of receptor solution was removed after each successive dose and analyzed using HPLC methodology. Each series of data represents the release of naftifine hydrochloride across the nail plate.

This Example compared the In Vitro Nail Permeation of two naftifine compositions: 5% Naftifine HCl and 10% Naftifine HCl. The raw data and comparative graphs are illustrated in Tables 3-5 shown below. Table 3 includes the standard deviation, and Table 4 includes the standard error. Table 5 is a summary of data for naftifine hydrochloride permeation across nail plates.

[image]

[image]

[image]

TABLE 5
Permeation Data

| | | | | | | |
|---|---|---|---|---|---|---|
| #1 | #2 | #3 | #4 | #5 | #6 | #7 |

5% Naftifine HCl Solution In-Vitro
(Micrograms Released Across the Nail Plate)

| | | | | | | | |
|---|---|---|---|---|---|---|---|
| NAIL #1 | 5.04 | 7.68 | 9.93 | 11.86 | 13.14 | 13.20 | 13.96 |
| NAIL #2 | 29.18 | 45.06 | 50.24 | 48.00 | 55.33 | 52.75 | 52.29 |
| NAIL #3 | 6.03 | 11.34 | 15.17 | 17.05 | 18.89 | 20.83 | 23.30 |
| NAIL #4 | 3.04 | 6.73 | 8.11 | 9.60 | 11.44 | 11.32 | 12.54 |
| NAIL #5 | 4.92 | 10.72 | 12.54 | 13.14 | 14.40 | 15.40 | 16.22 |
| MEAN | 9.64 | 16.31 | 19.20 | 19.93 | 22.64 | 22.70 | 23.66 |
| STD DEV | 10.97 | 16.19 | 17.56 | 15.92 | 18.48 | 17.17 | 16.53 |
| STD ERROR | 4.91 | 7.24 | 7.85 | 7.12 | 8.27 | 7.68 | 7.39 |

10% Naftifine HCl Solution In-Vitro
(Micrograms Released Across the Nail Plate)

| | | | | | | | |
|---|---|---|---|---|---|---|---|
| NAIL #1 | 1.30 | 3.63 | 5.59 | 6.74 | 7.20 | 7.42 | 7.21 |
| NAIL #2 | 1.73 | 4.10 | 6.07 | 7.03 | 7.47 | 7.53 | 7.42 |
| NAIL #3 | 4.18 | 7.26 | 10.13 | 11.68 | 12.32 | 13.22 | 13.50 |
| NAIL #4 | 7.36 | 11.40 | 14.77 | 15.40 | 15.62 | 15.34 | 14.80 |
| NAIL #5 | 23.20 | 31.67 | 38.22 | 40.36 | 39.17 | 36.83 | 34.97 |
| MEAN | 7.55 | 11.61 | 14.95 | 16.24 | 16.36 | 16.07 | 15.58 |
| STD DEV | 9.07 | 11.64 | 13.52 | 13.95 | 13.23 | 12.12 | 11.38 |
| STD ERROR | 4.06 | 5.20 | 6.05 | 6.24 | 5.92 | 5.42 | 5.09 |

The total mean amount of active naftifine hydrochloride transferred through the nail plate and into the receptor solution after seven applications was 23.66 μg for the 5% naftifine composition and 15.58 μg for the 10% naftifine composition. The release profiles for each composition were reproducible. The 5% naftifine hydrochloride solution consistently demonstrated an increased flux compared to the 10% composition.

1. A non-water soluble, film-forming composition which adheres to body tissue and forms a pharmaceutical carrier to provide localized delivery of an antifungal agent to a treatment site, said composition comprising:
(a) an alkyl cellulose present in about 0.5% (w/w) to about 3.0% (w/w) of the composition;
(b) a hydroxyalkyl cellulose present in about 2.0% (w/w) to about 10.0% (w/w) of the composition;
(c) a pharmaceutically acceptable polar protic solvent present in about 30.0% (w/w) to about 95.0% (w/w) of the composition;
(d) an antifungal agent selected from the group of naftifine, ciclopirox, terbinafine, pharmaceutically acceptable salts thereof, and combinations thereof, present in about 2.5% (w/w) to about 15% (w/w) of the composition;
(e) an glycol ether present in about 4.0% (w/w) to about 12.0% (w/w) of the composition;
(f) an antipruritic agent selected from the group of camphor, menthol, butamben picrate, metacresol, benzyl alcohol, camphorated metacresol, juniper tar, phenol, phenolate sodium, resorcinol, camphorated metacresol, carbolic acid, and combinations, present in about 1.0% (w/w) to about 5.0% (w/w) of the composition; and
(g) a solubility enhancing agent, a surfactant, a wetting agent, or a combination thereof, present in about 1.0% (w/w) to about 3.0% (w/w) of the composition.

2. The composition of claim 1, wherein the alkyl cellulose comprises ethyl cellulose, propyl cellulose, butyl cellulose, cellulose acetate, or a combination thereof.

3. The composition of claim 1, wherein the alkyl cellulose comprises ethyl cellulose.

4. The composition of claim 1, wherein the alkyl cellulose is present in about 0.75% (w/w) to about 1.25% (w/w) of the composition.

5. The composition of claim 1, wherein the hydroxyalkyl cellulose comprises hydroxybutyl cellulose, ethylhydroxyethyl cellulose, hydroxypropyl cellulose, or a combination thereof.

6. The composition of claim 1, wherein the hydroxyalkyl cellulose comprises hydroxypropyl cellulose.

7. The composition of claim 1, wherein the hydroxyalkyl cellulose is present in about 2.5% (w/w) to about 7.5% (w/w) of the composition.

8. The composition of claim 1, wherein the ratio of hydroxyalkyl cellulose to alkyl cellulose is about 1:1 to about 10:1.

9. The composition of claim 1, wherein the ratio of hydroxyalkyl cellulose to alkyl cellulose is about 2.5:1 to about 7.5:1.

10. The composition of claim 1, wherein the hydroxyalkyl cellulose and alkyl cellulose are present in about 2.5% (w/w) to about 15.0% (w/w) of the composition.

11. The composition of claim 1, wherein the hydroxyalkyl cellulose and alkyl cellulose are present in about 2.5% (w/w) to about 10.0% (w/w) of the composition.

12. The composition of claim 1, wherein the hydroxyalkyl cellulose and alkyl cellulose are present in about 2.5% (w/w) to about 8.0% (w/w) of the composition.

13. The composition of claim 1, wherein the pharmaceutically acceptable polar protic solvent comprises a straight-chained or branched (C₁-C₃₀)alkyl substituted with one or more hydroxyl groups, a (C₃-C₁₀)cycloalkyl substituted with one or more hydroxyl groups, water, or a combination thereof.

14. The composition of claim 13, wherein the straight-chained or branched (C₁-C₃₀)alkyl substituted with one or more hydroxyl groups comprises methanol, ethanol, iso-propanol, or tert-butanol.

15. The composition of claim 1, wherein the pharmaceutically acceptable polar protic solvent comprises a combination of ethanol and water.

16. The composition of claim 15, wherein the ethanol is present in about 30.0% (w/w) to about 90.0% (w/w) of the composition.

17. The composition of claim 15, wherein the water is present in about 3.0% (w/w) to about 15.0% (w/w) of the composition.

18. The composition of claim 1, wherein the antifungal agent comprises naftifine hydrochloride.

19. The composition of claim 18, wherein the naftifine hydrochloride is present in about 2.5% (w/w) to about 7.5% (w/w) of the composition.

20. The composition of claim 18, wherein the naftifine hydrochloride is present in about 7.5% (w/w) to about 15.0% (w/w) of the composition.

21. The composition of claim 1, wherein the glycol ether is selected from the group of ethylene glycol monopropyl ether (propoxyethanol), ethylene glycol monobutyl ether (butoxyethanol), diethylene glycol monomethyl ether (methoxydiglycol), diethylene glycol monoethyl ether (ethoxydiglycol), diethylene glycol monobutyl ether (butoxydiglycol), diethylene glycol monoisopropyl ether (isopropyldiglycol), diethylene glycol monoisobutyl ether (isobutyl diglycol), propylene glycol monomethyl ether, dipropylene glycol monomethyl ether (PPG-2 methyl ether), tripropylene glycol monomethyl ether (PPG-3 methyl ether), propylene glycol n-propyl ether, dipropylene glycol n-propyl ether (PPG-2 propyl ether), propylene glycol monobutyl ether, dipropylene glycol monobutyl ether (PPG-2 butyl ether), propylene glycol monoisobutyl ether, dipropylene glycol dimethyl ether, and combinations thereof.

22. The composition of claim 1, wherein the glycol ether comprises diethylene glycol monoethyl ether (DGME).

23. The composition of claim 1, wherein the glycol ether is present in about 6.0% (w/w) to about 10.0% (w/w) of the composition.

24. The composition of claim 1, wherein the antipruritic agent comprises benzyl alcohol.

25. The composition of claim 1, wherein the antipruritic agent is present in about 1.5% (w/w) to about 4.5% (w/w) of the composition.

26. The composition of claim 1, wherein the solubility enhancing agent, surfactant, wetting agent, or combination thereof, comprises sodium laureth ether sulfate (SLES).

27. The composition of claim 1, wherein the solubility enhancing agent, surfactant, wetting agent, or combination thereof, is present in about 1.5% (w/w) to about 2.5% (w/w) of the composition.

28. The composition of claim 1, further comprising a polymer having bioadhesive properties.

29. The composition of claim 28, wherein said polymer having bioadhesive properties comprises polyacrylic acid, polyvinylpyrrolidone, sodium carboxymethyl cellulose, copolymers of lactic and glycolic acids, polycaprolactone, polyorthoesters, polyphosphazene, cellulose acetate, polyvinyl acetate, polyisobutylene, or a combination thereof.

30. The composition of claim 1, further comprising a permeation enhancer.

31. The composition of claim 1, further comprising a component which acts to adjust the kinetics or erodability of the pharmaceutical carrier.

32. The composition of claim 31, wherein the component which acts to adjust the kinetics of erodability of the pharmaceutical carrier comprises a water soluble polymer, copolymers of lactic and glycolic acids, polycaprolactone, polyorthoesters, polyphosphazene, and mixtures thereof.

33. A non-water soluble, film-forming composition which adheres to body tissue and forms a pharmaceutical carrier to provide localized delivery of naftifine, or a pharmaceutically acceptable salt thereof, to a treatment site, said composition comprising:
(a) ethyl cellulose;
(b) hydroxypropyl cellulose;
(c) purified water;
(d) ethanol;
(e) naftifine, or a pharmaceutically acceptable salt thereof;
(f) diethylene glycol monoethyl ether (DGME);
(g) benzyl alcohol; and
(h) sodium laureth ether sulfate (SLES).

34. A non-water soluble, film-forming composition which adheres to body tissue and forms a pharmaceutical carrier to provide localized delivery of naftifine, or a pharmaceutically acceptable salt thereof, to a treatment site, said composition comprising:
(a) ethyl cellulose present in about 1.0% (w/w) of the composition;
(b) hydroxypropyl cellulose present in about 5.0% (w/w) of the composition;
(c) purified water present in about 6.0% (w/w) of the composition;
(d) ethanol 190 present in about 70.0% (w/w) of the composition;
(e) naftifine, or a pharmaceutically acceptable salt thereof, present in about 5.0% (w/w) of the composition;
(f) diethylene glycol monoethyl ether (DGME) present in about 8.0% (w/w) of the composition;
(g) benzyl alcohol present in about 3.0% (w/w) of the composition; and
(h) sodium laureth ether sulfate (SLES) present in about 2.0% (w/w) of the composition.

35. A non-water soluble, film-forming composition which adheres to body tissue and forms a pharmaceutical carrier to provide localized delivery of naftifine, or a pharmaceutically acceptable salt thereof, to a treatment site, said composition comprising:
(a) ethyl cellulose present in about 1.0% (w/w) of the composition;
(b) hydroxypropyl cellulose present in about 5.0% (w/w) of the composition;
(c) purified water present in about 6.0% (w/w) of the composition;
(d) ethanol 190 present in about 65.0% (w/w) of the composition;
(e) naftifine, or a pharmaceutically acceptable salt thereof, present in about 10.0% (w/w) of the composition;
(f) diethylene glycol monoethyl ether (DGME) present in about 8.0% (w/w) of the composition;
(g) benzyl alcohol present in about 3.0% (w/w) of the composition; and
(h) sodium laureth ether sulfate (SLES) present in about 2.0% (w/w) of the composition.

36. A non-water soluble, film-forming composition which adheres to body tissue and forms a pharmaceutical carrier to provide localized delivery of naftifine, or a pharmaceutically acceptable salt thereof, to a treatment site, said composition consisting essentially of:
(a) ethyl cellulose;
(b) hydroxypropyl cellulose;
(c) purified water;
(d) ethanol;
(e) naftifine, or a pharmaceutically acceptable salt thereof;
(f) diethylene glycol monoethyl ether (DGME);
(g) benzyl alcohol; and
(h) sodium laureth ether sulfate (SLES).

37. A non-water soluble, film-forming composition which adheres to body tissue and forms a pharmaceutical carrier to provide localized delivery of naftifine, or a pharmaceutically acceptable salt thereof, to a treatment site, said composition consisting essentially of:
(a) ethyl cellulose present in about 1.0% (w/w) of the composition;
(b) hydroxypropyl cellulose present in about 5.0% (w/w) of the composition;
(c) purified water present in about 6.0% (w/w) of the composition;
(d) ethanol 190 present in about 70.0% (w/w) of the composition;
(e) naftifine, or a pharmaceutically acceptable salt thereof, present in about 5.0% (w/w) of the composition;
(f) diethylene glycol monoethyl ether (DGME) present in about 8.0% (w/w) of the composition;
(g) benzyl alcohol present in about 3.0% (w/w) of the composition; and
(h) sodium laureth ether sulfate (SLES) present in about 2.0% (w/w) of the composition.

38. A non-water soluble, film-forming composition which adheres to body tissue and forms a pharmaceutical carrier to provide localized delivery of naftifine, or a pharmaceutically acceptable salt thereof, to a treatment site, said composition consisting essentially of:
(a) ethyl cellulose present in about 1.0% (w/w) of the composition;
(b) hydroxypropyl cellulose present in about 5.0% (w/w) of the composition;
(c) purified water present in about 6.0% (w/w) of the composition;
(d) ethanol 190 present in about 65.0% (w/w) of the composition;
(e) naftifine, or a pharmaceutically acceptable salt thereof, present in about 10.0% (w/w) of the composition;
(f) diethylene glycol monoethyl ether (DGME) present in about 8.0% (w/w) of the composition;
(g) benzyl alcohol present in about 3.0% (w/w) of the composition; and
(h) sodium laureth ether sulfate (SLES) present in about 2.0% (w/w) of the composition.

39. A method for inhibiting a fungus, the method comprising contacting the fungus with an effective amount of the composition of claim 1.

40. The method of claim 39 wherein the contacting is in vitro.

41. The method of claim 39 wherein the contacting is in vivo.

42. A method for treating a fungal infection in a mammal in need of such treatment, said method comprising contacting the fungal infection with an effective amount of the composition of claim 1.

43. The method of claim 42, wherein the fungal infection is present on a topical surface of the mammal.

44. The method of claim 42, wherein the fungal infection is present on the nail of the mammal, under the nail of the mammal, or a combination thereof.

45. The method of claim 42, wherein the nail is present on a toe of the mammal.

46. The method of claim 42, wherein the nail is present on a hand of the mammal.

47. The method of claim 42, wherein the contacting comprises spraying, dipping, or direct application by finger or swab.

48. The method of claim 42, wherein as the composition dries, it forms a hydrated film.

49. The method of claim 42, further comprising removing the composition from the mammal with a polar protic solvent.

50. The method of claim 49, wherein the polar protic solvent comprises a straight-chained or branched (C₁-C₃₀)alkyl substituted with one or more hydroxyl groups, a (C₃-C₁₀)cycloalkyl substituted with one or more hydroxyl groups, water, or a combination thereof.
